(19) United States
(12) Patent Application Publication
(10) Pub. No.: US 2024/0138757 A1
(43) Pub. Date: May 2, 2024

(54) PELVIC INCLINATION ESTIMATION DEVICE, ESTIMATION SYSTEM, PELVIC INCLINATION ESTIMATION METHOD, AND RECORDING MEDIUM

(71) Applicant: NEC Corporation, Tokyo (JP)

(72) Inventors: Zhenwei WANG, Tokyo (JP); Chenhui HUANG, Tokyo (JP); Kenichiro FUKUSHI, Tokyo (JP); Kazuki IHARA, Tokyo (JP); Fumiyuki NIHEY, Tokyo (JP); Hiroshi KAJITANI, Tokyo (JP); Yoshitaka NOZAKI, Tokyo (JP); Kentaro NAKAHARA, Tokyo (JP)

(73) Assignee: NEC Corporation, Tokyo (JP)

(21) Appl. No.: 18/410,330

(22) Filed: Jan. 11, 2024

Related U.S. Application Data
(63) Continuation of application No. 18/203,303, filed on May 30, 2023.

(30) Foreign Application Priority Data
Jun. 8, 2022 (JP) 2022-092756

(51) Int. Cl. A61B 5/00; A61B 5/11; G16H 40/63

(52) U.S. Cl. CPC A61B 5/4571; A61B 5/112; A61B 5/7278; G16H 40/63

(57) ABSTRACT

Provided is a pelvic inclination estimation device including a communication unit that acquires feature amount data including a feature amount to be used for estimation of a pelvic inclination, the feature amount being extracted from a gait waveform of a spatial acceleration and a spatial angular velocity included in sensor data related to movement of a foot of a subject, a storage unit that stores an estimation model that outputs an estimation value related to the pelvic inclination according to an input of the feature amount included in the feature amount data, an estimation unit that inputs a feature amount included in the acquired feature amount data to the estimation model and estimate a pelvic inclination of the subject according to the estimation value output from the estimation model, and an output unit that outputs information associated to the pelvic inclination of the subject.

This application is a Continuation of U.S. application Ser. No. 18/203,303 filed on May 30, 2023, which is based upon and claims the benefit of priority from Japanese Patent Application No. 2022-092756, filed on Jun. 8, 2022, the disclosure of which is incorporated herein in its entirety by reference.

### TECHNICAL FIELD

The present disclosure relates to a pelvic inclination estimation device and the like that estimate a pelvic inclination that is an index related to movement of a waist.

### BACKGROUND ART

With growing interest in healthcare, services that provide information corresponding to a gait have attracted attention. For example, a technique for analyzing a gait using sensor data measured by a sensor mounted on footwear such as shoes has been developed. Features associated with a gait event related to physical conditions appear in the time-series data of the sensor data. The physical conditions of the subject can be estimated by analyzing the gait data including the features associated with the gait event.

Front-back, lateral, and vertical inclinations (also referred to as pelvic inclinations) of the pelvis (waist) during walking are indices indicating the shake and movement of the waist. A pelvic inclination is used as an index for visualization of walking and evaluation of walking stability. If the pelvic inclination can be estimated with high accuracy by analyzing the gait data, it is possible to provide a service according to the need for healthcare.

Patent Literature 1 (JP 2020-151470 A) discloses a walking evaluation device that evaluates walking ability of a user. The device of Patent Literature 1 calculates a plurality of gait indices related to a walking state using a plurality of pieces of gait data acquired from a subject. In the method of Patent Literature 1, a gait score of a subject is calculated using the gait data acquired by an acceleration sensor attached to the waist of the subject. In the method of Patent Literature 1, a harmonic ratio, which is one of gait indices, is calculated from acceleration waveforms in the vertical direction, the lateral direction, and the front-back direction measured by an acceleration sensor attached to the waist of the subject.

Patent Literature 2 (WO 2022/038664 A1) discloses a calculation device that calculates step lengths of both right and left feet using sensor data based on movement of a foot measured by a sensor installed on a foot portion of a pedestrian. The device of Patent Literature 2 calculates the step lengths of the left and right feet according to the gait event timing appearing in the gait waveform of the traveling-direction acceleration and the traveling direction trajectory.

Patent Literature 3 (WO 2016/031313 A1) discloses physical condition detection device that specifies physical conditions of a user using a photographed image including a walking movement of the user photographed by a photographing device. The device of Patent Literature 3 extracts a region in which the user appears in the captured image, and analyzes the walking movement of the user in the real space based on depth data inside the extracted region. Patent Literature 3 discloses an item including a lateral inclination of a pelvis as walking characteristics indicating the properties of walking movement.

In the method of Patent Literature 1, one of indices related to the movement of the waist is measured using acceleration measured by an acceleration sensor attached to the waist. In daily life, sensors worn on the waist can limit free actions. If the position of the attached sensor deviates, the measurement accuracy decreases. Therefore, in the method of Patent Literature 1, it is not possible to easily measure the index related to the movement of the waist with high accuracy in daily life.

In the method of Patent Literature 2, the step lengths of both left and right feet are calculated using sensor data based on the movement of the foot. Patent Literature 2 does not disclose estimating the pelvic inclination using sensor data based on the movement of the foot.

In the method of Patent Literature 3, the walking movement corresponding to the walking characteristics including the lateral inclination of the pelvis is analyzed using the photographed image. Patent Literature 3 does not disclose estimating the pelvic inclination in conjunction with the movement of the foot. In the method of Patent Literature 3, it is necessary to photograph the user with a camera in order to analyze the walking movement including the walking characteristics.

An object of the present disclosure is to provide a pelvic inclination estimation device and the like that can easily estimate the pelvic inclination that is an index related to the movement of the waist with high accuracy in daily life.

### SUMMARY

A pelvic inclination estimation device according to an aspect of the present disclosure includes a communication unit that acquires feature amount data including a feature amount to be used for estimation of a pelvic inclination that is an index related to movement of a waist, the feature amount being extracted from a gait waveform of a spatial acceleration and a spatial angular velocity included in sensor data related to movement of a foot of a subject, a storage unit that stores an estimation model that outputs an estimation value related to the pelvic inclination according to an input of the feature amount included in the feature amount data, an estimation unit that inputs a feature amount included in the acquired feature amount data to the estimation model and estimate a pelvic inclination of the subject according to the estimation value related to the pelvic inclination output from the estimation model, and an output unit that outputs information associated to the pelvic inclination of the subject.

A pelvic inclination estimation method according to one aspect of the present disclosure includes acquiring feature amount data including a feature amount to be used for estimation of a pelvic inclination that is an index related to movement of a waist, the feature amount being extracted from sensor data related to movement of a foot of a subject, storing an estimation model that outputs an estimation value related to the pelvic inclination according to an input of the feature amount data, inputting the acquired feature amount data to the estimation model and estimating a pelvic inclination of the subject according to the estimation value related to the pelvic inclination output from the estimation model, and outputting information associated to the pelvic inclination of the subject.

A program according to an aspect of the present disclosure causes a computer to execute processing of acquiring feature amount data including a feature amount to be used for estimation of a pelvic inclination that is an index related to movement of a waist, the feature amount being extracted from sensor data related to movement of a foot of a subject, processing of storing an estimation model that outputs an estimation value related to the pelvic inclination according to an input of the feature amount data, processing of inputting the acquired feature amount data to the estimation model and estimating a pelvic inclination of the subject according to the estimation value related to the pelvic inclination output from the estimation model, and processing of outputting information associated to the pelvic inclination of the subject.

### BRIEF DESCRIPTION OF THE DRAWINGS

Exemplary features and advantages of the present invention will become apparent from the following detailed description when taken with the accompanying drawings in which:

FIG. 1 is a block diagram illustrating an example of a configuration of an estimation system according to a first example embodiment;

FIG. 2 is a block diagram illustrating an example of a configuration of a measurement device included in the estimation system according to the first example embodiment;

FIG. 3 is a conceptual diagram illustrating an arrangement example of the measurement device according to the first example embodiment;

FIG. 4 is a conceptual diagram for explaining an example of a relationship between a local coordinate system and a global coordinate system set in the measurement device according to the first example embodiment;

FIG. 5 is a conceptual diagram for explaining a human body surface;

FIG. 6 is a conceptual diagram for explaining a gait cycle;

FIG. 7 is a graph for explaining an example of time-series data of sensor data measured by the measurement device according to the first example embodiment;

FIG. 8 is a diagram for explaining an example of normalization of gait waveform data by the measurement device according to the first example embodiment;

FIG. 9 is a conceptual diagram for explaining a feature amount cluster from which a feature amount data generation unit of the measurement device according to the first example embodiment extracts a feature amount;

FIG. 10 is a block diagram illustrating an example of a configuration of a pelvic inclination estimation device included in the estimation system according to the first example embodiment;

FIG. 11 is a graph for explaining an example of a difference in pelvic inclination about the left-right axis;

FIG. 12 is a graph for explaining another example of the difference in pelvic inclination about the left-right axis;

FIG. 13 is a graph for explaining an example of a difference in pelvic inclination about the traveling axis;

FIG. 14 is a graph for explaining an example of a difference in pelvic inclination about the vertical axis;

FIG. 15 is a conceptual diagram for explaining learning of the estimation model used by the pelvic inclination estimation device included in the estimation system according to the first example embodiment;

FIG. 16 is a table summarizing an example of input data to be used for estimation of the pelvic inclination about the axis of travel by the pelvic inclination estimation device included in the estimation system according to the first example embodiment;

FIG. 17 is a table summarizing an example of input data to be used for estimation of the pelvic inclination about the left-right axis by the pelvic inclination estimation device included in the estimation system according to the first example embodiment;

FIG. 18 is a flowchart for explaining an example of the operation of the measurement device included in the estimation system according to the first example embodiment;

FIG. 19 is a flowchart for explaining an example of the operation of the pelvic inclination estimation device included in the estimation system according to the first example embodiment;

FIG. 20 is a conceptual diagram for explaining an application example of the estimation system according to the first example embodiment;

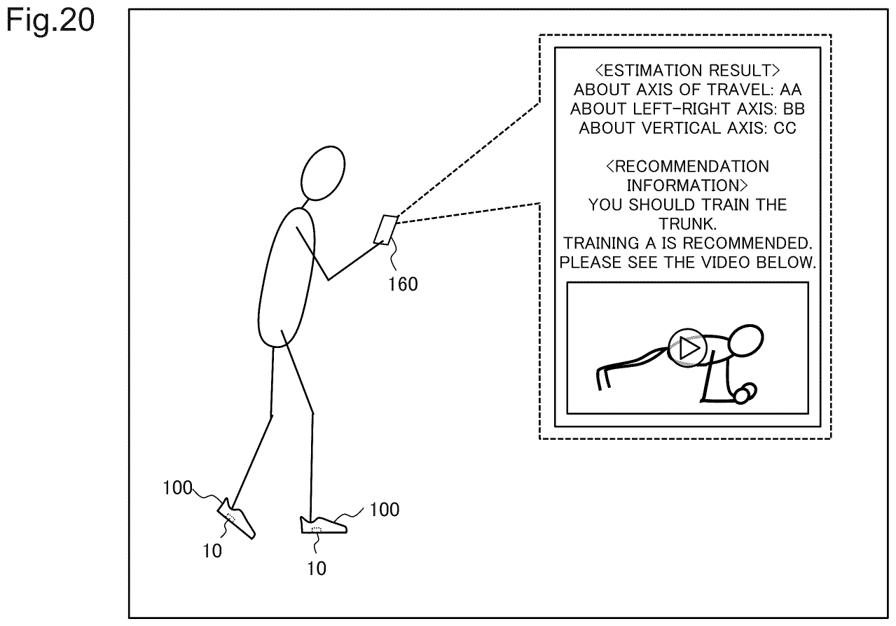

FIG. 21 is a conceptual diagram for explaining an application example of the estimation system according to the first example embodiment;

FIG. 22 is a block diagram illustrating an example of a configuration of a pelvic inclination estimation device according to a second example embodiment; and

FIG. 23 is a block diagram illustrating an example of a hardware configuration that executes processing of each example embodiment.

### EXAMPLE EMBODIMENT

Example embodiments of the present invention will be described below with reference to the drawings. In the following example embodiments, technically preferable limitations are imposed to carry out the present invention, but the scope of this invention is not limited to the following description. In all drawings used to describe the following example embodiments, the same reference numerals denote similar parts unless otherwise specified. In addition, in the following example embodiments, a repetitive description of similar configurations or arrangements and operations may be omitted.

### First Example Embodiment

First, an estimation system according to a first example embodiment will be described with reference to the drawings. The estimation system according to the present example embodiment measures sensor data related to movement of a foot according to a gait of a user using a measurement device mounted on footwear. The estimation system of the present example embodiment estimates a pelvic inclination which is an index related to the movement of the waist using the measured sensor data. Pelvic inclination corresponds to an inclination angle of the pelvis during walking.

The left and right feet are connected to the pelvis through the lower thigh and the thigh. Hip and knee joints are located between the left and right feet and the pelvis, but the periodicity of the pelvis and the waist during walking is similar. Therefore, there is a phase in which the movement of the left and right feet and the movement of the waist interlock with each other. In the present example embodiment, the pelvic inclination which is an index related to the movement of the waist is estimated using the sensor data related to the movement of the foot. Details of the pelvic inclination will be described later.

(Configuration)

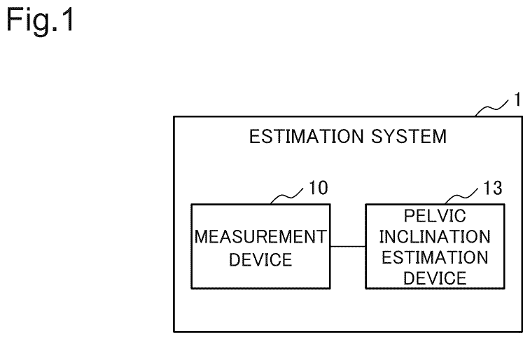

FIG. 1 is a block diagram illustrating an example of a configuration of an estimation system 1 according to the present example embodiment. The estimation system 1 includes a measurement device 10 and a pelvic inclination estimation device 13. In the present example embodiment, an example in which the measurement device 10 and the pelvic inclination estimation device 13 are configured as separate hardware will be described. For example, the measurement device 10 is installed on footwear or the like of a subject (user) who is an estimation target of pelvic inclination. For example, the function of the pelvic inclination estimation device 13 is installed in a mobile terminal carried by the subject (user). Hereinafter, configurations of the measurement device 10 and the pelvic inclination estimation device 13 will be individually described.

[Measurement Device]

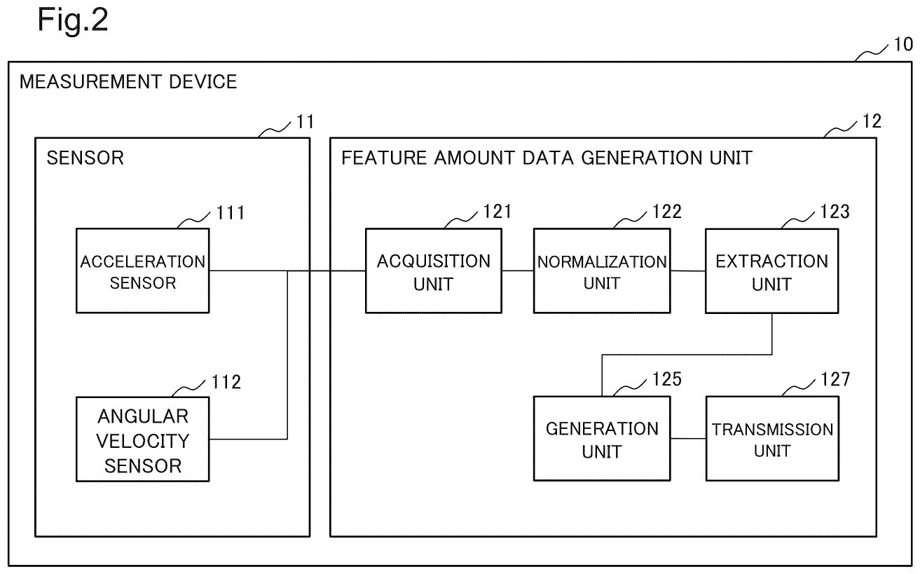

FIG. 2 is a block diagram illustrating an example of a configuration of the measurement device 10. The measurement device 10 includes a sensor 11 and a feature amount data generation unit 12. In the present example embodiment, an example in which the sensor 11 and the feature amount data generation unit 12 are integrated will be described. The sensor 11 and the feature amount data generation unit 12 may be provided as separate devices.

As illustrated in FIG. 2, the sensor 11 includes an acceleration sensor 111 and an angular velocity sensor 112. FIG. 2 illustrates an example in which the acceleration sensor 111 and the angular velocity sensor 112 are included in the sensor 11. The sensor 11 may include sensors other than the acceleration sensor 111 and the angular velocity sensor 112. Sensors other than the acceleration sensor 111 and the angular velocity sensor 112 that can be included in the sensor 11 will not be described.

The acceleration sensor 111 is a sensor that measures accelerations (also referred to as spatial accelerations) in three axial directions. The acceleration sensor 111 measures acceleration (also referred to as spatial acceleration) as a physical quantity related to the movement of the foot. The acceleration sensor 111 outputs the measured acceleration to the feature amount data generation unit 12. For example, a sensor of a piezoelectric type, a piezoresistive type, a capacitance type, or the like can be used as the acceleration sensor 111. The measurement method of the sensor used as the acceleration sensor 111 is not particularly limited as long as the sensor can measure acceleration.

The angular velocity sensor 112 is a sensor that measures an angular velocity (also referred to as a spatial angular velocity) about three axes. The angular velocity sensor 112 measures an angular velocity (also referred to as a spatial angular velocity) as a physical quantity related to the movement of the foot. The angular velocity sensor 112 outputs the measured angular velocity to the feature amount data generation unit 12. For example, a sensor of a vibration type, a capacitance type, or the like can be used as the angular velocity sensor 112. The measurement method of the sensor used as the angular velocity sensor 112 is not particularly limited as long as the sensor can measure an angular velocity.

The sensor 11 is, for example, an inertial measurement device that measures acceleration and angular velocity. An example of the inertial measurement device is an inertial measurement unit (IMU). The IMU includes the acceleration sensor 111 that measures accelerations in three axial directions and the angular velocity sensor 112 that measures angular velocities about the three axes. The sensor 11 may be an inertial measurement device such as a vertical gyro (VG) or an attitude heading Reference System (AHRS). The sensor 11 may be GPS/INS (Global Positioning System/Inertial Navigation System). The sensor 11 may be a device other than the inertial measurement device as long as it can measure a physical quantity related to the movement of the foot.

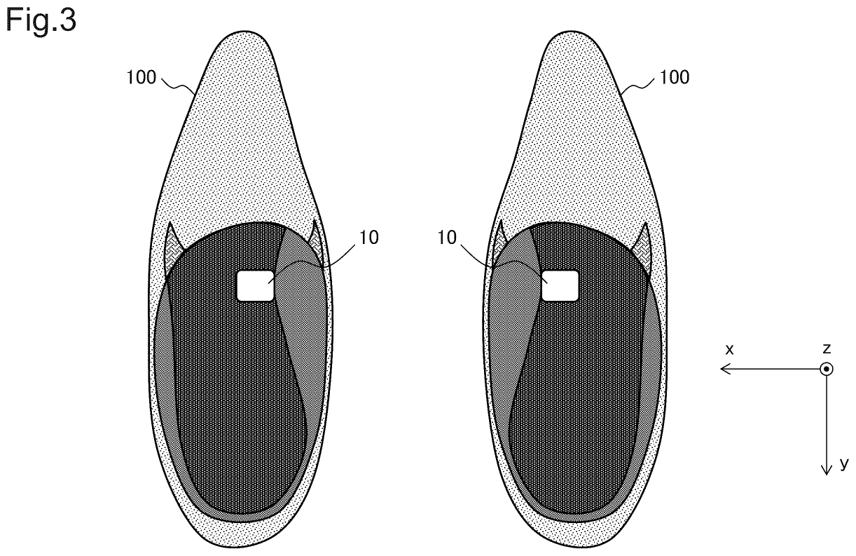

FIG. 3 is a conceptual diagram illustrating an example in which the measurement device 10 is arranged in the shoes 100 of both feet. In the example of FIG. 3, the measurement device 10 is installed at a position corresponding to the back side of the arch of foot. The measurement device 10 may be installed at a position other than the back side of the arch of the foot as long as the sensor data related to the movement of the foot can be measured. For example, the measurement device 10 is disposed in an insole inserted into the shoe 100. The measurement device 10 may be disposed on the bottom surface of the shoe 100. The measurement device 10 may be embedded in the main body of the shoe 100. The measurement device 10 may be detachable from the shoe 100 or may not be detachable from the shoe 100. The measurement device 10 may be installed on a sock worn by the user or a decorative article such as an anklet worn by the user. The measurement device 10 may be directly attached to the foot or may be embedded in the foot. FIG. 3 illustrates an example in which the measurement device 10 is installed on the shoes 100 of both feet. The measurement device 10 may be installed on the shoe 100 of one foot.

In the example of FIG. 3, a local coordinate system including the x-axis in the horizontal direction, the y-axis in the traveling direction, and the z-axis in the vertical direction is set with reference to the measurement device 10 (sensor 11). The left side of the x-axis is positive, the rear side of the y-axis is positive, and the upper side of the z-axis is positive. The direction of the axis set in the sensor 11 may be the same for the left and right feet, or may be different for the left and right feet. For example, in a case where the sensors 11 produced with the same specifications are arranged in the left and right shoes 100, the vertical directions (directions in the Z-axis direction) of the sensors 11 arranged in the left and right shoes 100 are the same. In this case, the three axes of the local coordinate system set in the sensor data derived from the left foot and the three axes of the local coordinate system set in the sensor data derived from the right foot are the same on the left and right. In a case where the sensors 11 produced with different specifications on the left and right are arranged in the shoes 100, the vertical directions (directions in the Z-axis direction) of the sensors 11 arranged on the left and right shoes 100 may be different.

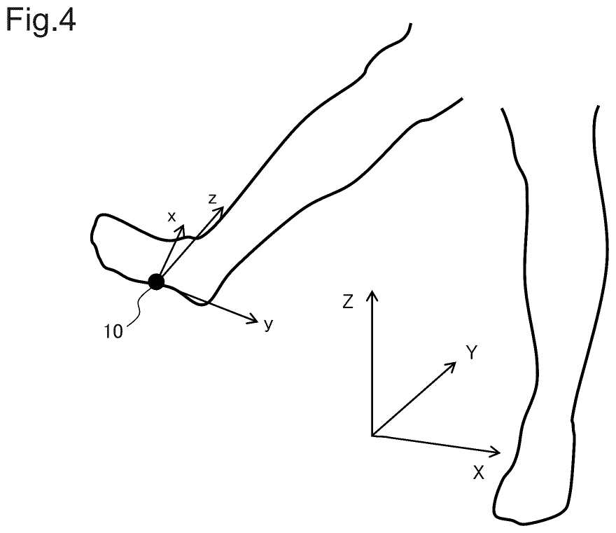

FIG. 4 is a conceptual diagram for explaining a local coordinate system (x-axis, y-axis, z-axis) set in the measurement device 10 (sensor 11) installed on the back side of the arch of the foot and a global coordinate system (X-axis, Y-axis, Z-axis) set with respect to the ground. In the global coordinate system (X-axis, Y-axis, Z-axis), in a state where the user facing the traveling direction is upright, the lateral direction of the user is set to the X-axis direction (the leftward direction is positive), the direction of the back surface of the user is set to the Y-axis direction (the rearward direction is positive), and the gravity direction is set to the Z-axis direction (the vertically upward direction is positive). The example of FIG. 4 conceptually illustrates the relationship between the local coordinate system (x-axis, y-axis, z-axis) and the global coordinate system (X-axis, Y-axis, Z-axis), and does not accurately illustrate the relationship between the local coordinate system and the global coordinate system that varies depending on the walking of the user.

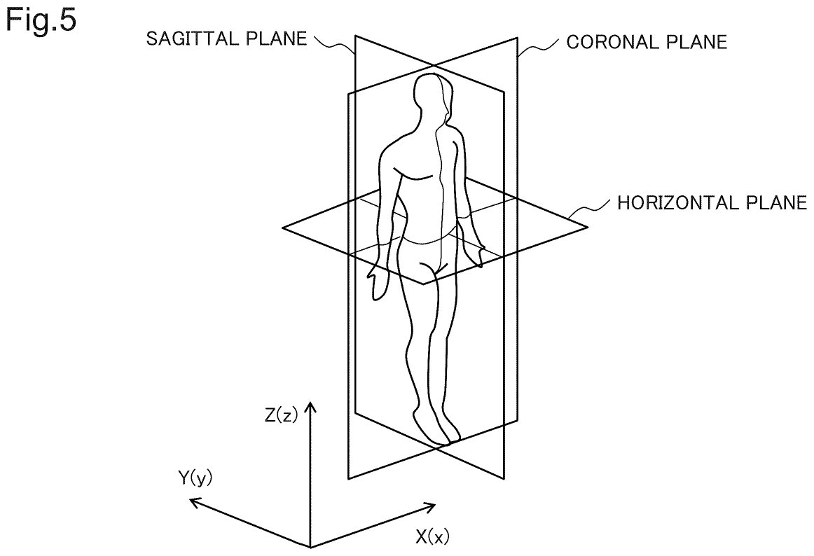

FIG. 5 is a conceptual diagram for explaining a surface (also referred to as a human body surface) set for the human body. In the present example embodiment, a sagittal plane dividing the body into left and right, a coronal plane dividing the body into front and rear, and a horizontal plane dividing the body horizontally are defined. As illustrated in FIG. 5, the global coordinate system and the local coordinate system coincide with each other in a state in which the user is upright with the center line of the foot oriented in the traveling direction. In the present example embodiment, rotation in the sagittal plane with the x-axis as the rotation axis is defined as roll, rotation in the coronal plane with the y-axis as the rotation axis is defined as pitch, and rotation in the horizontal plane with the z-axis as the rotation axis is defined as yaw. A rotation angle in the sagittal plane with the x-axis as a rotation axis is defined as a roll angle, a rotation angle in the coronal plane with the y-axis as a rotation axis is defined as a pitch angle, and a rotation angle in the horizontal plane with the z-axis as a rotation axis is defined as a yaw angle. In the following description, the x-axis, the y-axis, and the z-axis are expressed as three axes.

As illustrated in FIG. 2, the feature amount data generation unit 12 (also referred to as a feature amount data generation device) includes an acquisition unit 121, a normalization unit 122, an extraction unit 123, a generation unit 125, and a transmission unit 127. For example, the feature amount data generation unit 12 includes a microcomputer or a microcontroller that performs overall control and data processing of the measurement device 10. For example, the feature amount data generation unit 12 includes a central processing unit (CPU), a random access memory (RAM), a read only memory (ROM), a flash memory, and the like. The feature amount data generation unit 12 controls the acceleration sensor 111 and the angular velocity sensor 112 to measure the angular velocity and the acceleration. For example, the feature amount data generation unit 12 may be mounted on a mobile terminal (not illustrated) carried by a subject (user). In this case, the sensor 11 may be provided with a communication function, and the sensor data transmitted from the sensor 11 may be received by the mobile terminal on which the feature amount data generation unit 12 is mounted.

The acquisition unit 121 acquires accelerations in three axial directions from the acceleration sensor 111. The acquisition unit 121 acquires angular velocities about three axes from the angular velocity sensor 112. For example, the acquisition unit 121 performs analog-to-digital conversion (AD conversion) on the acquired physical quantities (analog data) such as an angular velocity and an acceleration. The physical quantity (analog data) measured by the acceleration sensor 111 and the angular velocity sensor 112 may be converted into digital data in each of the acceleration sensor 111 and the angular velocity sensor 112. The acquisition unit 121 outputs the converted digital data (also referred to as sensor data) to the normalization unit 122. The acquisition unit 121 may be configured to store the sensor data in a storage unit (not illustrated). The sensor data includes at least acceleration data converted into digital data and angular velocity data converted into digital data. The acceleration data includes acceleration vectors in three axial directions. The angular velocity data includes angular velocity vectors about three axes. The acceleration data and the angular velocity data are associated with acquisition time of the data. The acquisition unit 121 may add correction such as a mounting error, temperature correction, and linearity correction to the acceleration data and the angular velocity data.

The normalization unit 122 acquires sensor data from the acquisition unit 121. The normalization unit 122 extracts time-series data (also referred to as gait waveform data) for one gait cycle from the time-series data of the acceleration in the three axial directions and the angular velocity about the three axes included in the sensor data. The normalization unit 122 normalizes (also referred to as first normalization) the time of the extracted gait waveform data for one gait cycle to a gait cycle of 0 to 100% (percent). The timing such as 1% or 10% included in the 0 to 100% gait cycle is also referred to as a gait phase. The normalization unit 122 normalizes (also referred to as second normalization) the first normalized gait waveform data for one gait cycle so that the stance phase becomes 60% and the swing phase becomes 40%. The stance phase is a period in which at least a part of the back side of the foot is in contact with the ground. The swing phase is a period in which the back side of the foot is separated from the ground. When the gait waveform data is subjected to the second normalization, it is possible to reduce the influence of the deviation of the gait phase that may occur in each gait cycle.

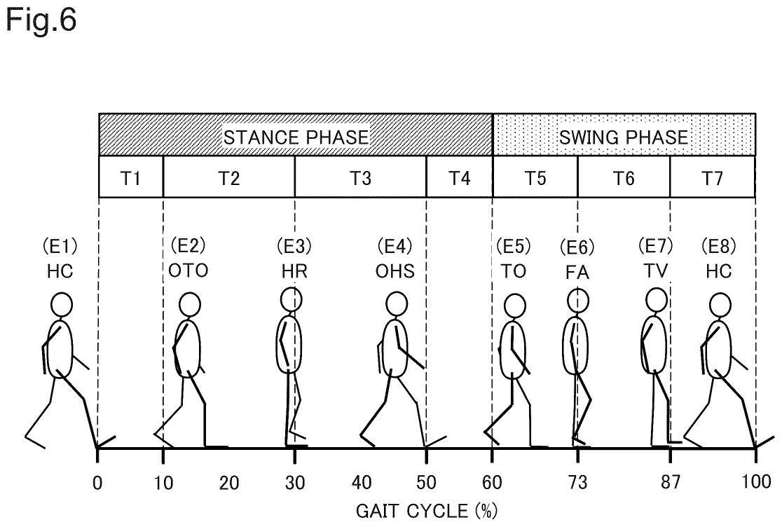

FIG. 6 is a conceptual diagram for explaining one gait cycle with the right foot as a reference. One gait cycle based on the left foot is also similar to that of the right foot. The horizontal axis of FIG. 6 is one gait cycle of the right foot with a time point at which the heel of the right foot lands on the ground as a start point and a time point at which the heel of the right foot next lands on the ground as an end point. The horizontal axis in FIG. 6 is first normalized with one gait cycle as 100%. The horizontal axis of FIG. 6 is second normalized so that the stance phase is 60% and the swing phase is 40%. The one gait cycle of one foot is roughly divided into a stance phase in which at least a part of the back side of the foot is in contact with the ground and a swing phase in which the back side of the foot is separated from the ground. The stance phase is further subdivided into an initial stance period T1, a mid-stance period T2, a terminal stance period T3, and a pre-swing period T4. The swing phase is further subdivided into an initial swing period T5, a mid-swing period T6, and a terminal swing period T7. FIG. 6 is an example, and does not limit the periods constituting one gait cycle, the names of these periods, and the like.

As illustrated in FIG. 6, during walking, a plurality of events (also referred to as gait events) occur. E1 represents an event (heel-contact: HC) in which the heel of the right foot touches the ground. E2 represents an event (opposite toe-off: OTO) in which the toe of the left foot is separated from the ground with the sole of the right foot in contact with the ground (OTO). E3 represents an event (heel-rise: HR) in which the heel of the right foot lifts with the sole of the right foot in contact with the ground. E4 is an event (opposite heel-strike: OHS) in which the heel of the left foot is in contact with the ground. E5 represents an event (toe-off: TO) in which the toe of the right foot is separated from the ground with the sole of the left foot in contact with the ground. E6 represents an event (foot-adjacent: FA) in which the left foot and the right foot cross with the sole of the left foot in contact with the ground. E7 represents an event (tibia-vertical: TV) that the tibia of the right foot is approximately vertical to the ground with the sole of the left foot in contact with the ground. E8 represents an event (heel-contact: HC) in which the heel of the right foot touches the ground. E8 corresponds to the end point of the gait cycle starting from E1 and corresponds to the start point of the next gait cycle. FIG. 6 is an example, and does not limit events that occur during walking or names of these events.

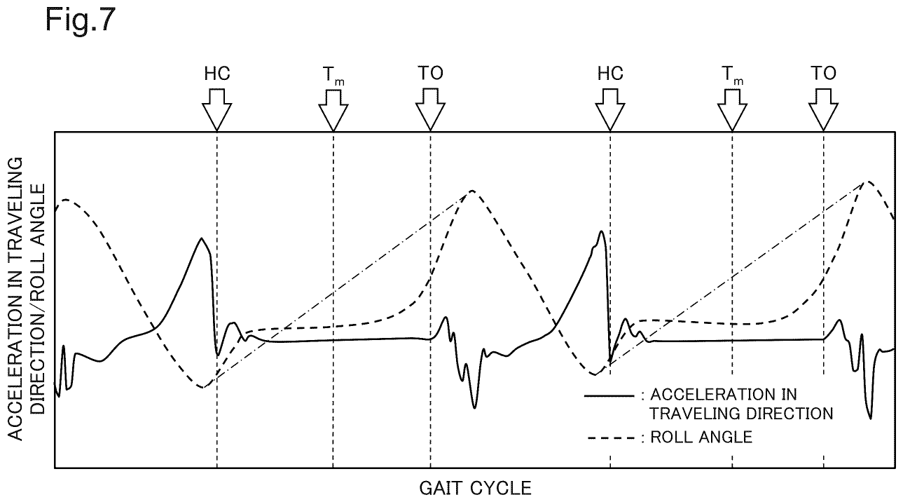

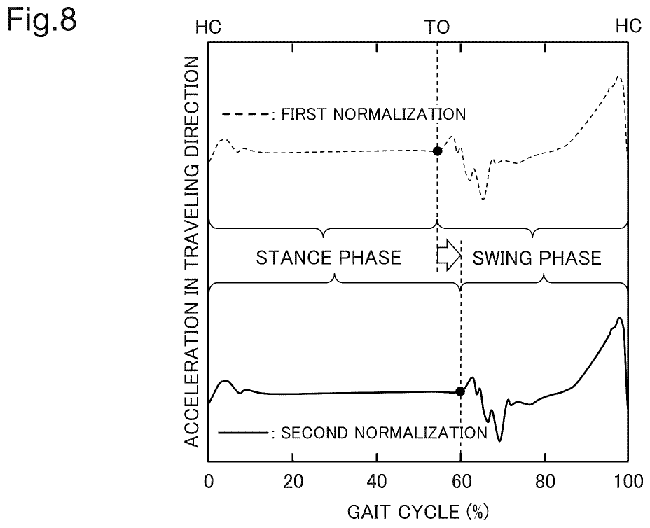

FIG. 7 is a diagram for explaining an example of detecting the heel-contact HC and the toe-off TO from the time-series data (solid line) of the traveling-direction acceleration (Y-direction acceleration). The timing of the heel-contact HC is the timing of the minimum peak immediately after the maximum peak appearing in the time-series data of the traveling-direction acceleration (Y-direction acceleration). The maximum peak serving as a mark of the timing of the heel-contact HC corresponds to the maximum peak of the gait waveform data for one gait cycle. A section between the consecutive heel-contacts HC is one gait cycle. The timing of the toe-off TO is the rising timing of the maximum peak appearing after the period of the stance phase in which a fluctuation does not appear in the time-series data of the traveling-direction acceleration (Y-direction acceleration). FIG. 8 also illustrates time-series data (broken line) of the roll angle (angular velocity about the X-axis). The timing at the midpoint between the timing of the minimum roll angle and the timing of the maximum roll angle corresponds to the timing Tm of the transition from the mid-stance period T2 to the terminal stance period T3. The timing Tm of the transition from the mid-stance period T2 to the terminal stance period T3 substantially coincides with the timing of the heel-rise HR. The parameters (also referred to as gait parameters) used for the estimation of the physical conditions can be obtained with reference to the timing Tm of the transition from the mid-stance period T2 to the terminal stance period T3.

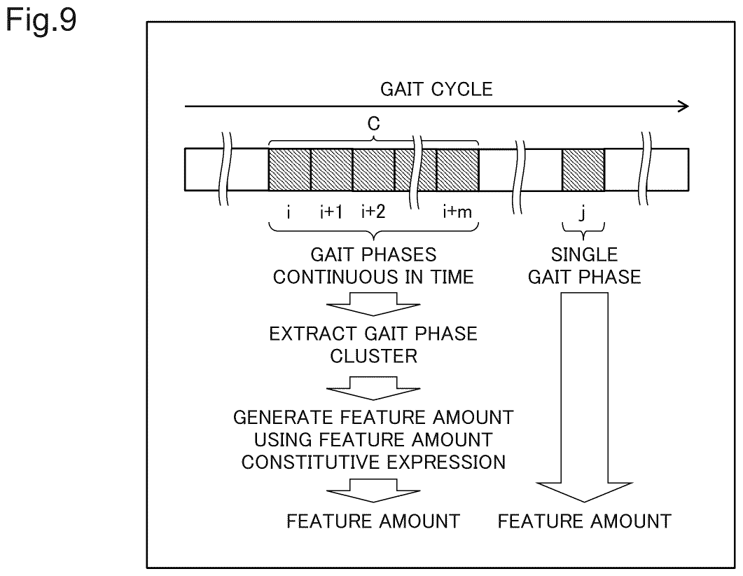

FIG. 8 is a diagram for explaining an example of normalization of gait waveform data. The normalization unit 122 detects the heel-contact HC and the toe-off TO from the time-series data of the traveling-direction acceleration (Y-direction acceleration). The normalization unit 122 extracts a section between consecutive heel-contacts HC as gait waveform data for one gait cycle. The normalization unit 122 converts the horizontal axis (time axis) of the gait waveform data for one gait cycle into a gait cycle of 0 to 100% by the first normalization. In FIG. 9, the gait waveform data after the first normalization is indicated by a broken line. In the gait waveform data (broken line) after the first normalization, the timing of the toe-off TO deviates from 60%.

In the example of FIG. 8, the normalization unit 122 normalizes a section from the heel-contact HC in which the gait phase is 0% to the toe-off TO subsequent to the heel-contact HC to 0 to 60%. The normalization unit 122 normalizes a section from the toe-off TO to the heel-contact HC in which the gait phase subsequent to the toe-off TO is 100% to 60 to 100%. As a result, the gait waveform data for one gait cycle is normalized to a section (stance phase) in which the gait cycle is 0 to 60% and a section (swing phase) in which the gait cycle is 60 to 100%. In FIG. 9, the gait waveform data after the second normalization is indicated by a solid line. In the gait waveform data (solid line) after the second normalization, the timing of the toe-off TO coincides with 60%. For example, when the times of the stance phase and the swing phase and the ratio thereof are verified, the second normalization may be omitted.

FIGS. 7 to 8 illustrate examples in which the gait waveform data for one gait cycle is extracted/normalized based on the traveling-direction acceleration (Y-direction acceleration). Regarding the acceleration/angular velocity other than the traveling-direction acceleration (Y-direction acceleration), the normalization unit 122 extracts/normalizes gait waveform data for one gait cycle according to the gait cycle of the traveling-direction acceleration (Y-direction acceleration). The normalization unit 122 may generate time-series data of angles about three axes by integrating time-series data of angular velocities about the three axes. In this case, the normalization unit 122 also extracts/normalizes the gait waveform data for one gait cycle according to the gait cycle of the traveling-direction acceleration (Y-direction acceleration) with respect to the angles about the three axes.

The normalization unit 122 may extract/normalize the gait waveform data for one gait cycle based on acceleration/angular velocity other than the traveling-direction acceleration (Y-direction acceleration) (not illustrated). For example, the normalization unit 122 may detect the heel-contact HC and the toe-off TO from the time-series data of the vertical-direction acceleration (Z-direction acceleration). The timing of the heel-contact HC is a timing of a steep minimum peak appearing in the time-series data of the vertical-direction acceleration (Z-direction acceleration). At the timing of the steep minimum peak, the value of the vertical-direction acceleration (Z-direction acceleration) becomes substantially zero. The minimum peak serving as a mark of the timing of the heel-contact HC corresponds to the minimum peak of the gait waveform data for one gait cycle. A section between the consecutive heel-contacts HC is one gait cycle. The timing of the toe-off TO is a timing of an inflection point in the middle of gradually increasing after the time-series data of the vertical-direction acceleration (Z-direction acceleration) passes through a section with a small fluctuation after the maximum peak immediately after the heel-contact HC. The normalization unit 122 may extract/normalize the gait waveform data for one gait cycle based on both the traveling-direction acceleration (Y-direction acceleration) and the vertical-direction acceleration (Z-direction acceleration). The normalization unit 122 may extract/normalize the gait waveform data for one gait cycle based on acceleration, angular velocity, angle, and the like other than the traveling-direction acceleration (Y-direction acceleration) and the vertical-direction acceleration (Z-direction acceleration).

The extraction unit 123 acquires gait waveform data for one gait cycle normalized by the normalization unit 122. The extraction unit 123 extracts a feature amount to be used for estimation of the pelvic inclination from the gait waveform data for one gait cycle. The extraction unit 123 extracts a feature amount (also referred to as a cluster feature amount) for each gait phase cluster from the gait phase clusters obtained by integrating temporally consecutive gait phases based on a preset condition. The gait phase cluster includes at least one gait phase. The gait phase cluster may also be composed of a single gait phase. The gait waveform data and the gait phase from which the feature amount to be used for estimation of the pelvic inclination is extracted will be described later.

FIG. 9 is a conceptual diagram for explaining extraction of a feature amount for estimating a pelvic inclination from gait waveform data for one gait cycle. For example, the extraction unit 123 extracts temporally consecutive gait phases i to i+m as the gait phase cluster C (i and m are natural numbers). In the present example embodiment, an example in which the gait phase cluster C used for estimation of the pelvic inclination is selected by correlation analysis using statistic parametric mapping will be described. For example, the gait phase cluster C may be selected by Pearson's correlation analysis.

In the example of FIG. 9, the gait phase cluster C includes m gait phases (components). That is, the number of gait phases (components) (also referred to as the number of components) constituting the gait phase cluster C is m. FIG. 9 illustrates an example in which the number of gait phases has an integer value, but the number of gait phases may be subdivided into decimal places. When the number of gait phases is subdivided into decimal places, the number of components of the gait phase cluster C is a number corresponding to the number of data points in the section of the gait phase cluster. The extraction unit 123 extracts a feature amount from each of the gait phases i to i+m. In a case where the gait phase cluster C is composed of a single gait phase j, the extraction unit 123 extracts a feature amount from the single gait phase j (j is a natural number).

The generation unit 125 applies the feature amount constitutive expression to the feature amount extracted from each of the gait phases constituting the gait phase cluster to generate the feature amount (cluster feature amount) of the gait phase cluster. The cluster feature amount is also referred to as a first feature amount. The feature amount constitutive expression is a preset calculation formula for generating the feature amount of the gait phase cluster. For example, the feature amount constitutive expression is a calculation formula related to four arithmetic operations. For example, the cluster feature amount calculated using the feature amount constitutive equation is an integral average value, an arithmetic average value, an inclination, a variation, or the like of the feature amount in each gait phase included in the gait phase cluster. For example, the generation unit 125 applies a calculation formula for calculating the inclination or variation of the feature amount extracted from each of the gait phases constituting the gait phase cluster as the feature amount constitutive formula. For example, in a case where the gait phase cluster is composed of a single gait phase, it is not possible to calculate the inclination or variation, and thus, it is sufficient to use a feature amount constituent equation for calculating an integral average value, an arithmetic average value, or the like. For example, in a case where the gait phase cluster is composed of a single gait phase, the feature amount extracted from the gait phase may be set as the cluster feature amount.

The generation unit 125 calculates parameters (also referred to as gait parameters) related to the gait. The generation unit 125 calculates the gait parameters using the feature amount derived from the gait waveform data. The gait parameters include features to be used for estimation of physical conditions. The estimation system 1 may be configured to calculate gait parameters on the side of the pelvic inclination estimation device 13. Hereinafter, examples of the gait parameters calculated by the generation unit 125 will be listed. The following gait parameters are merely examples, and do not cover all parameters including the features of the gait. In the present example embodiment, among the following gait parameters, those having a high correlation in estimation of the pelvic inclination are selected. Details of the method for calculating the gait parameters will be omitted.

Examples of the gait parameters include a stride, a walking pitch, a walking speed, a contact angle, a take-off angle, an outward turning distance (diversion amount), and a toe direction (inward/outward turning). The stride is a distance between the toes of both feet in a state in which one step is taken with one of the left and right feet and the toes land on the ground. The walking pitch is the number of steps within a predetermined time, and is used for calculating the walking speed. The walking speed is a moving speed in one gait cycle. The walking speed may be a value averaged in a plurality of gait cycles. The contact angle is an angle (posture angle) of the sole with respect to the ground in a state where the heel is in contact with the ground. The contact angle is an angle (posture angle) of the sole with respect to the ground in a state where the toes are in contact with the ground. The outward turning distance is a distance between a foot and a straight line indicating a moving route at a timing when the foot is farthest from the moving route of one foot in one gait cycle. The toe direction is an angle between a straight line indicating a movement route of one foot in one gait cycle and a center line of the foot in a landed state.

Examples of the gait parameters include a roll angle, a foot lift height, a maximum angular velocity in a plantarflexion direction, a maximum angular velocity in a dorsiflexion direction, a maximum speed, a maximum acceleration of a foot during the swing phase, and a cadence. For example, the roll angle at the heel-contact or the toe-off is used as the gait parameters. The foot lift height corresponds to the height of the foot in the vertical direction. For example, the maximum angular velocity in the plantarflexion direction and the maximum angular velocity in the dorsiflexion direction during the swing phase are used as the gait parameters. For example, the maximum speed during the swing phase is used as the gait parameters. The maximum acceleration of the foot during the swing phase is the maximum value of the vertical-direction acceleration of the foot during the swing phase, and relates to the rise of the waist according to the interlocking of the movement of the foot and the waist. The cadence corresponds to the number of steps in 60 seconds.

Examples of the gait parameters include a stance time, a swing time, a double support time (DST), a load time, a plantar contact time, and a kicking time. The stance time is a time corresponding to the period of the stance phase. The swing time is a time corresponding to the period of the swing phase. The DST corresponds to a double-leg support period during walking. The DST includes a DST1 corresponding to a double-leg support period after the heel-contact and a DST2 corresponding to a double-leg support period immediately before kicking. The load time is a time during which a load is applied to the sole of the foot. The load time corresponds to a time from heel-contact to plantar contact. The plantar contact time is a time during which the main surface of the plantar surface is in contact with the ground. The plantar contact time corresponds to the time from the plantar contact to the heel-off. The kicking time is a time from application of a load to the main surface of the sole of the foot to kicking of the foot. The kicking time corresponds to the time from the plantar contact to the toe-off.

The transmission unit 127 outputs feature amount data including the cluster feature amount generated by the generation unit 125. In a case where the gait parameters are used for estimation of the pelvic inclination, the transmission unit 127 outputs the feature amount data including the cluster feature amounts and the gait parameters. The transmission unit 127 transmits the feature amount data to the pelvic inclination estimation device 13. For example, the transmission unit 127 transmits the feature amount data to the pelvic inclination estimation device 13 via wireless communication. For example, the transmission unit 127 is configured to transmit the feature amount data to the pelvic inclination estimation device 13 via a wireless communication function (not illustrated) conforming to a standard such as Bluetooth (registered trademark) or WiFi (registered trademark). The communication function of the transmission unit 127 may conform to a standard other than Bluetooth (registered trademark) or WiFi (registered trademark).

[Pelvic Inclination Estimation Device]

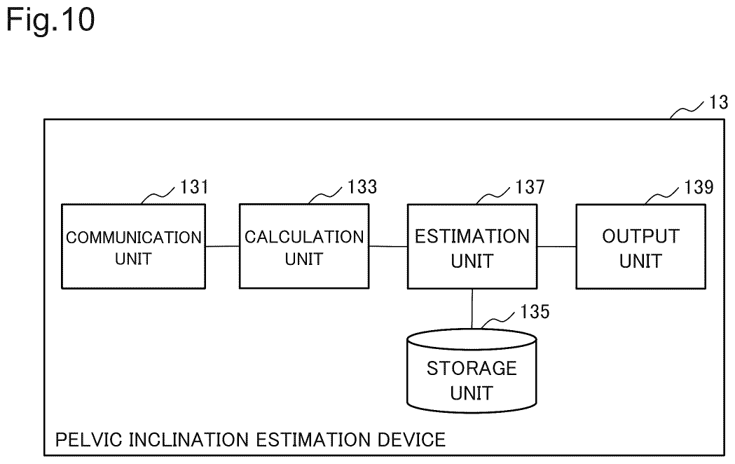

FIG. 10 is a block diagram illustrating an example of a configuration of the pelvic inclination estimation device 13. The pelvic inclination estimation device 13 includes a communication unit 131, a calculation unit 133, a storage unit 135, an estimation unit 137, and an output unit 139.

The communication unit 131 acquires the feature amount data from the measurement device 10. The communication unit 131 outputs the received data to the calculation unit 133. The communication unit 131 may receive the feature amount data from the measurement device 10 via a wire such as a cable, or may receive the feature amount data from the measurement device 10 via wireless communication. For example, the communication unit 131 is configured to receive the feature amount data from the measurement device 10 via a wireless communication function (not illustrated) conforming to a standard such as Bluetooth (registered trademark) or WiFi (registered trademark). The communication function of the communication unit 131 may conform to a standard other than Bluetooth (registered trademark) or WiFi (registered trademark).

The calculation unit 133 acquires the feature amount data. The calculation unit 133 calculates input data to be used for estimation of the pelvic inclination using the cluster feature amounts and the gait parameters included in the acquired feature amount data. The calculation unit 133 calculates an average value of the first feature amounts for both feet used for estimation of the pelvic inclination. The calculation unit 133 calculates the absolute value of the difference of the first feature amounts for both feet used for estimation of the pelvic inclination. The calculation unit 133 calculates an average value of the gait parameters for both feet used for estimation of the pelvic inclination. The calculation unit 133 calculates the absolute value of the difference of the gait parameters for both feet used for estimation of the pelvic inclination. Hereinafter, the absolute value of the difference is also referred to as a difference. The average value or difference of the first feature amounts/gait parameters for both feet calculated by the calculation unit 133 is also referred to as a second feature amount. The second feature amount is used for estimation of the pelvic inclination. Instead of the average value or difference of the first feature amounts or the gait parameters included in the feature amount data generated by the measurement device 10, the first feature amounts or the gait parameters may be directly used for estimation of the pelvic inclination. In this case, the calculation unit 133 can be omitted.

The storage unit 135 stores an estimation model for estimating the pelvic inclination. The estimation model outputs the estimation result related to the pelvic inclination according to the input of the input data calculated by the calculation unit 133. The storage unit 135 stores estimation models learned for a plurality of subjects. In a case where the attribute of the subject is used for estimation, the storage unit 135 stores the attribute of the subject. For example, the attribute of the subject includes gender, age, weight, height, and the like of the subject. The pelvic inclination estimation device 13 estimates the pelvic inclinations in the three axes of the traveling axis, the left-right axis, and the vertical axis. The attribute of the subject varies depending on the inclination axis of the pelvic inclination to be estimated.

The estimation model is stored in the storage unit 135 at the time of factory shipment of a product, calibration before the user uses the estimation system, or the like. For example, the estimation system 1 may be configured to use an estimation model stored in a storage device such as an external server. In that case, the estimation system 1 may be configured such that the estimation model is used via an interface (not illustrated) connected to the storage device.

The estimation unit 137 acquires, from the calculation unit 133, input data to be used for estimation of the pelvic inclination. When the feature amount data generated by the measurement device 10 is used as it is, the estimation unit 137 acquires the feature amount data as input data. In a case where the attribute of the subject is used for estimation, the estimation unit 137 acquires the attribute of the subject from the storage unit 135.

The estimation unit 137 estimates the pelvic inclination using the acquired input data. In the present example embodiment, an example of estimating the variation width of the pelvic inclination which is the difference between the maximum value and the minimum value of the pelvic inclination in one gait cycle will be described. The estimation unit 137 inputs input data to the estimation model stored in the storage unit 135. The estimation unit 137 outputs the estimation result of the pelvic inclination output from the estimation model. In a case where an estimation model stored in an external storage device constructed in a cloud, a server, or the like is used, the estimation unit 137 is configured to use the estimation model via an interface (not illustrated) connected to the storage device.

Pelvic inclination is an index indicating inclination of the pelvis. The estimation unit 137 estimates the pelvic inclination about the three axes of the left-right axis (roll), the axis of travel (pitch), and the vertical axis (yaw). Pelvic inclination with respect to the left-right axis corresponds to pelvic inclination in the sagittal plane. Pelvic inclination with respect to the traveling axis corresponds to pelvic inclination in the coronal plane. Pelvic inclination with respect to the vertical axis corresponds to pelvic inclination in the horizontal plane. Pelvic inclination about the left-right axis (roll) corresponds to inclination of the body in the front-back direction (in the sagittal plane) about the pelvic position. Pelvic inclination about the axis of travel (pitch) corresponds to inclination of the body in the vertical direction (in the coronal plane) about the pelvic position. Pelvic inclination about the vertical axis (yaw) corresponds to rotation (in the horizontal plane) of the body about the pelvic position. Using the pelvic inclination, the movement of the subject, which cannot be grasped only by the movement of the foot, can be grasped.

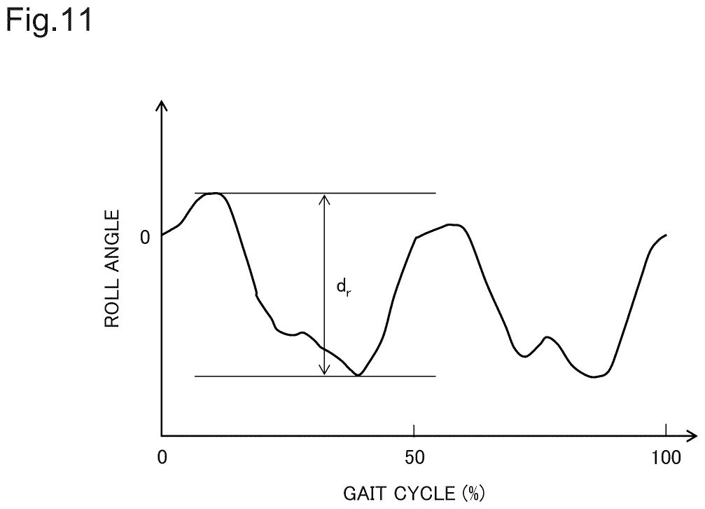

Regarding the pelvic inclination about the left-right axis (roll), one of the anteflexion/retroflexion in the sagittal plane is positive and the other is negative. FIG. 11 is a graph illustrating an example of time-series data of the pelvic inclination about the left-right axis (roll). In the graph of FIG. 11, time-series data of the pelvic inclination about the left-right axis (roll) is associated with the gait cycle. In one gait cycle, a difference d r between the maximum value and the minimum value of the pelvic inclination about the left-right axis (roll) corresponds to the variation width of the pelvic inclination about the left-right axis (roll).

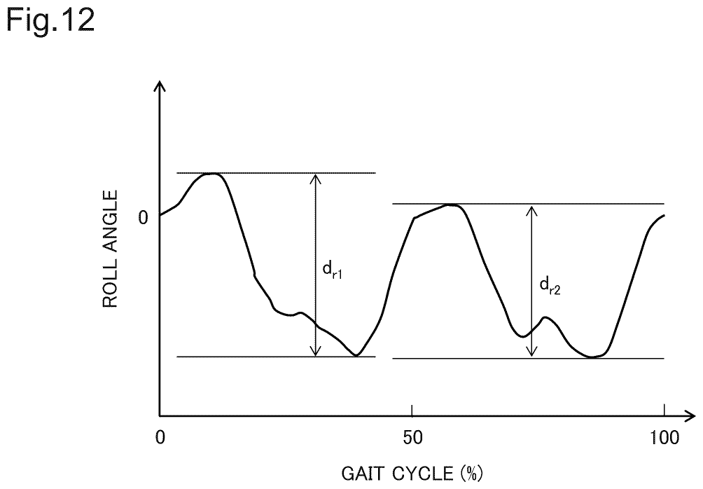

FIG. 12 is a graph illustrating another example of the time-series data of the pelvic inclination about the left-right axis (roll). In the graph of FIG. 12, time-series data of the pelvic inclination about the left-right axis (roll) is associated with the gait cycle. In the time-series data of the pelvic inclination about the left-right axis (roll), a variation width (difference dᵣ₁) of the preceding amplitude and a variation width (difference (1,2) of the subsequent amplitude appear. In the example of FIG. 12, in one gait cycle, the variation width (difference dᵣ₁) of the preceding amplitude and the variation width (difference (1,2) of the subsequent amplitude are separately estimated. For example, the variation width (difference dᵣ₁) of the preceding amplitude and the variation width (difference (1,2) of the subsequent amplitude are estimated using different estimation models associated to the respective variation widths.

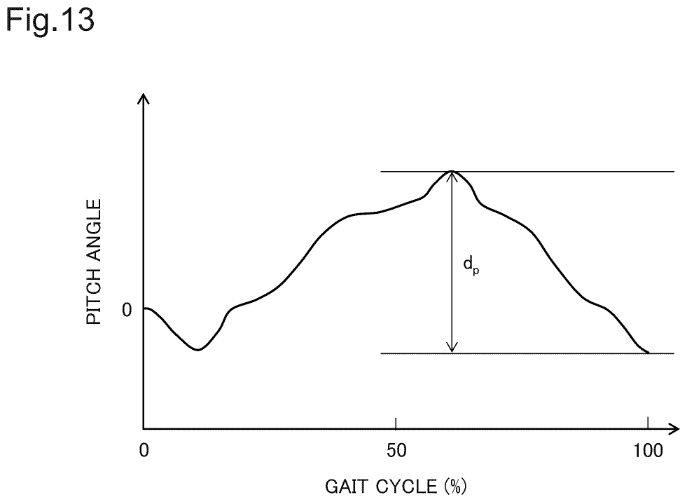

Regarding the pelvic inclination about the axis of travel (pitch), either the leftward inclination or the rightward inclination in the coronal plane is positive, and the other is negative. FIG. 13 is a graph illustrating an example of time-series data of the pelvic inclination about the axis of travel (pitch). In the graph of FIG. 13, time-series data of the pelvic inclination about the axis of travel (pitch) is associated with the gait cycle. In one gait cycle, a difference dₚ between the maximum value and the minimum value of the pelvic inclination about the axis of travel (pitch) corresponds to the variation width of the pelvic inclination about the axis of travel (pitch).

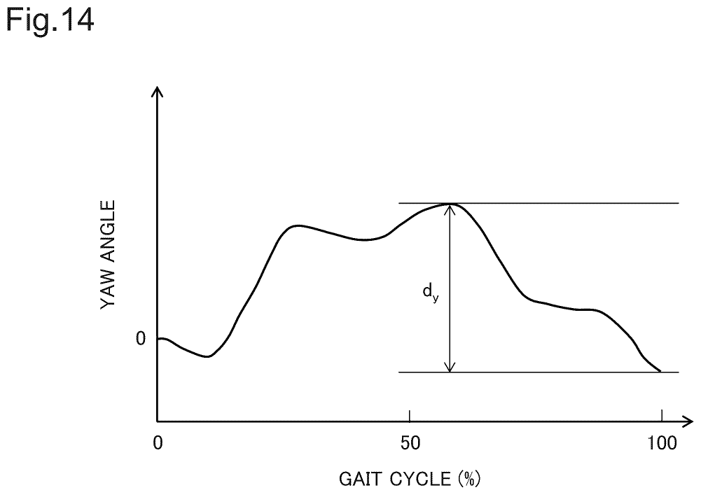

Regarding the pelvic inclination about the vertical axis, either clockwise or counterclockwise about the waist in the horizontal plane is positive, and the other is negative. FIG. 14 is a graph illustrating an example of time-series data of the pelvic inclination about the vertical axis. In the graph of FIG. 14, the time-series data of the pelvic inclination about the vertical axis is associated with the gait cycle. In one gait cycle, the difference d_{y} between the maximum value and the minimum value of the pelvic inclination about the vertical axis corresponds to the variation width of the pelvic inclination about the vertical axis.

The output unit 139 outputs the estimation result of the pelvic inclination by the estimation unit 137. For example, the output unit 139 displays the estimation result of the pelvic inclination on the screen of the mobile terminal of the subject (user). For example, the output unit 139 outputs the estimation result to an external system or the like that uses the estimation result. The use of the information related to the pelvic inclination output from the pelvic inclination estimation device 13 is not particularly limited.

For example, the pelvic inclination estimation device 13 is connected to an external system or the like constructed in a cloud or a server via a mobile terminal (not illustrated) carried by a subject (user). The mobile terminal (not illustrated) is a portable communication device. For example, the mobile terminal is a portable communication device having a communication function, such as a smartphone, a smart watch, or a mobile phone. For example, the pelvic inclination estimation device 13 is connected to a mobile terminal via a wire such as a cable. For example, the pelvic inclination estimation device 13 is connected to a mobile terminal via wireless communication. For example, the pelvic inclination estimation device 13 is connected to a mobile terminal via a wireless communication function (not illustrated) conforming to a standard such as Bluetooth (registered trademark) or WiFi (registered trademark). The communication function of the pelvic inclination estimation device 13 may conform to a standard other than Bluetooth (registered trademark) or WiFi (registered trademark). The estimation result of the pelvic inclination may be used by an application installed in the mobile terminal. In that case, the mobile terminal executes processing using the estimation result by application software or the like installed in the mobile terminal.

[Learning Example]

Next, a learning example of an estimation model used for estimation of the pelvic inclination by the pelvic inclination estimation device 13 will be described with reference to a verification result related to a correlation between a difference in pelvic inclination and feature amount data. Hereinafter, a verification example performed on forty-five subjects will be described. In the following verification example, the correlation between the measured value and the estimation value of the pelvic inclination during walking was verified. In the present verification example, a subject wearing a smart apparel and a shoe on which the measurement device 10 is mounted was allowed to walk twice on a straight path of 5 m. An IMU that measures a spatial acceleration and a spatial angular velocity is mounted on the waist of the smart apparel. The measured values were derived using the measured values of the spatial acceleration and the spatial angular velocity of the waist of the subject. The prediction value is an estimation value estimated using sensor data measured by the measurement device 10 mounted on the shoe worn by the subject at the same time as the measurement of the measured value. The correlation between the measured value and the estimation value is evaluated by a correlation coefficient.

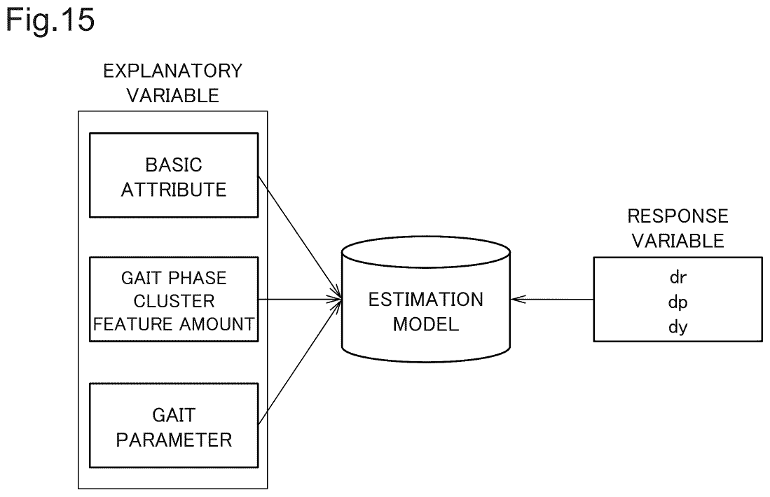

FIG. 15 is a conceptual diagram for explaining an example of learning of an estimation model used for estimation of the pelvic inclination. For learning of the estimation model, explanatory variables and response variables related to a plurality of subjects are used. As the explanatory variable, the attribute of the subject, the cluster feature amount generated according to the gait of the subject, and the gait parameters are used. As the response variable, a measured value of the pelvic inclination simultaneously measured at the time of measuring the sensor data for generating the cluster feature amounts and the gait parameters is used. The measured value of the pelvic inclination includes a difference dr about the left-right axis, a difference d p about the axis of travel, and a difference d_{y} about the vertical axis. For example, the measured value of the pelvic inclination is derived using the measured value measured by the IMU attached to the waist of the subject. For example, the estimation model is a multiple regression model constructed using the feature amount selected by the Leave-one-subject-out LASSO method.

For example, the estimation model is constructed by learning using a linear regression algorithm. For example, the estimation model is constructed by learning using a support vector machine (SVM) algorithm. For example, the estimation model is constructed by learning using a Gaussian Process Regression (GPR) algorithm. For example, the estimation model is constructed by learning using a random forest (RF) algorithm. The estimation model may be constructed by unsupervised learning that classifies subjects who are generation sources of the feature amount data according to the feature amount data. The algorithm used for learning the estimation model is not particularly limited.

The estimation model may be constructed by learning using gait waveform data (sensor data) for one gait cycle as an explanatory variable. For example, the estimation model is constructed by supervised learning in which the acceleration in the three axial directions, the angular velocity about the three axes, and the gait waveform data of the angle (posture angle) about the three axes are used as explanatory variables and the measured value of the pelvic inclination that is the estimation target is used as an objective variable.

<About Left-Right Axis (Roll)>

The average value or difference of the first feature amounts/gait parameters of both feet is used for estimation of the difference in the pelvic inclination about the left-right axis. The body weight is used as the attribute of the user in estimation of the difference in the pelvic inclination about the left-right axis. In the present example embodiment, an example in which the first feature amount is not used will be described.

The first feature amounts/gait parameters of both feet are used for estimation of the difference in pelvic inclination about the left-right axis. For example, an average value of both feet of the stride length, the roll angle at the toe-off, and the maximum speed during the swing phase is used as the second feature amount. For example, a difference of both feet of the maximum diversion amount, the roll angle at the heel-contact, the minimum value in the swing phase, and the swing peak is used as the second feature amount. In this verification, regarding the estimation of the difference in pelvic inclination about the left-right axis, the correlation coefficient between the measured value and the estimation value was 0.4580.

Regarding the estimation of the difference in the pelvic inclination about the left-right axis, as illustrated in FIG. 12, an example in which the difference dᵣ₁ of the preceding amplitude and the difference dᵣ₂ of the subsequent amplitude are separately estimated in one gait cycle is also exemplified. In this case, regarding the preceding amplitude difference dᵣ₁, the correlation coefficient between the measured value and the estimation value was 0.4945. Regarding the subsequent amplitude difference dᵣ₂, the correlation coefficient between the measured value and the estimation value was 0.6842. As described above, regarding the estimation of the difference of the pelvic inclination about the left-right axis, the correlation coefficient was larger when the difference dᵣ₁ of the preceding amplitude and the difference dᵣ₂ appearing in the latter half were separately estimated.

<About Axis of Travel (Pitch)>

The average value or difference of the first feature amounts/gait parameters of both feet is used for estimation of the difference in the pelvic inclination about the axis of travel. Age and weight are used as attributes of the user in estimation of the difference in pelvic inclination about the axis of travel.

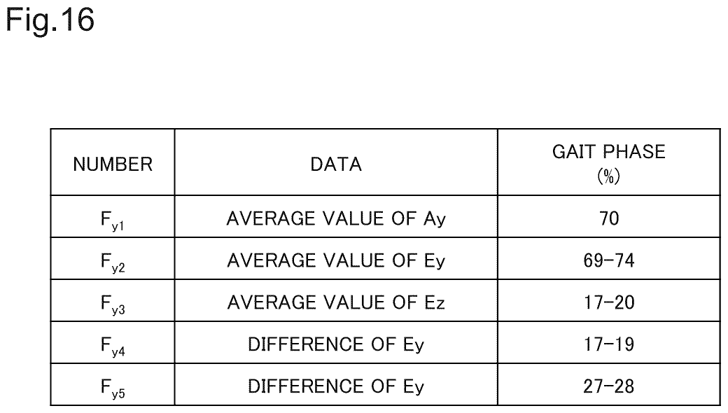

FIG. 16 is a table summarizing an example of the second feature amount of both feet used for estimation of the difference in the pelvic inclination about the axis of travel. Regarding the estimation of the difference in pelvic inclination about the axis of travel, the average value of both feet in the traveling-direction acceleration A_{y}, the angle E_{y} about the axis of travel, and the angle E about the vertical axis is used as the second feature amount. Regarding the traveling-direction acceleration A_{y}, the second feature amount F_{y1} in the section of the gait phase of 70% is used for estimation. Regarding the angle (pitch angle) E about the axis of travel, the second feature amount F_{y2} in the section of the gait phase of 69 to 74% is used for estimation. Regarding the angle (yaw angle) E about the vertical axis, the second feature amount F_{y3} in the section of the gait phase of 17 to 20% is used for estimation. Regarding the estimation of the difference in the pelvic inclination about the axis of travel, the difference of both feet of the angle (pitch angle) E about the axis of travel is used as the second feature amount. Regarding the angle (pitch angle) E_{y} about the axis of travel, the second feature amount F_{y4} in the section of the gait phase of 17 to 19% and the first feature amount F_{y5} in the section of the gait phase of 27 to 28% are used for estimation.

A plurality of gait parameters is used for estimation of the difference in pelvic inclination about the axis of travel. For example, the average value of both feet of the maximum plantarflexion angle, the maximum diversion amount, the pronation/supination angle, the roll angle at the heel-contact, the load time, and the plantar contact time, and DST2 is used as the second feature amount. For example, a difference of both feet of a walking speed, a maximum dorsiflexion angle, a pronation/supination angle, cadence, a swing time, a plantar contact time, DST1, and a minimum value in the swing phase is used as the second feature amount. In this verification, regarding the estimation of the difference in pelvic inclination about the axis of travel, the correlation coefficient between the measured value and the estimation value was 0.7468.

<About Vertical Axis (Yaw)>

Regarding the estimation of the difference in pelvic inclination about the vertical axis, an average value or a difference of gait parameters of both feet is used. The body weight is used as the attribute of the user in estimation of the difference in the pelvic inclination about the vertical axis.

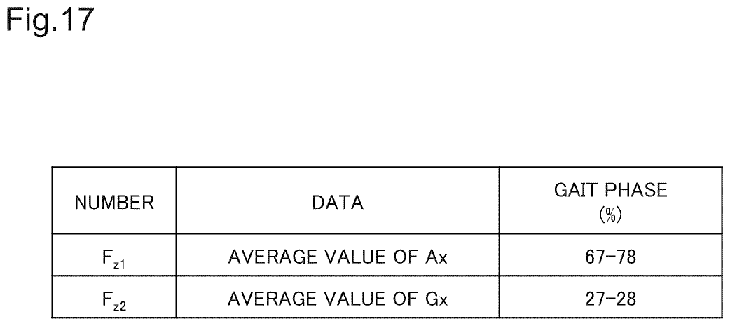

FIG. 17 is a table summarizing an example of the average value or difference of the first feature amounts of both feet used for estimation of the difference in the pelvic inclination about the vertical axis. Regarding the estimation of the difference in the pelvic inclination about the vertical axis, the average value of both feet of the horizontal-direction acceleration Aₓ and the angular velocity Gₓ about the left-right axis is used as the second feature amount. Regarding the horizontal-direction acceleration Aₓ, the first feature amount F_{z1} in the section of the gait phase of 67 to 78% is used for estimation. Regarding the angular velocity Gₓ about the left-right axis, the first feature amount F_{z2} in the section of the gait phase of 27 to 28% is used for estimation.

A plurality of gait parameters is used for estimation of a difference in pelvic inclination about the vertical axis. For example, an average value of both feet of the stride length and the kicking time is used as the second feature amount. For example, a difference of both feet of the maximum plantarflexion angle, the pronation/supination angle, the stance time, the swing peak, and the maximum speed during the swing phase is used as the second feature amount. In this verification, regarding the estimation of the difference in pelvic inclination about the vertical axis, the correlation coefficient between the measured value and the estimation value was 0.5630.

(Operation)

Next, an operation of the estimation system 1 will be described with reference to the drawings. Here, the measurement device 10 and the pelvic inclination estimation device 13 included in the estimation system 1 will be individually described. Regarding the measurement device 10, the operation of the feature amount data generation unit 12 included in the measurement device 10 will be described.

[Measurement Device]

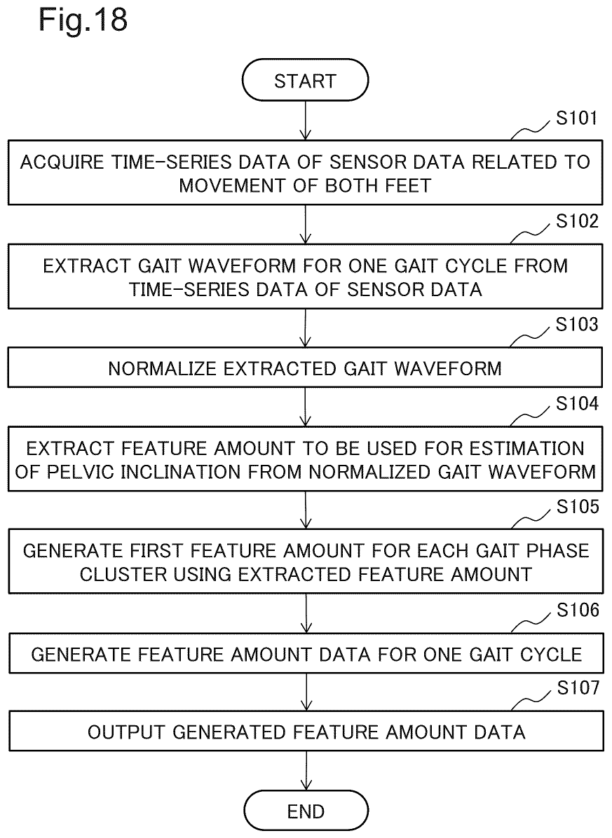

FIG. 18 is a flowchart for explaining the operation of the feature amount data generation unit 12 included in the measurement device 10. In the description according to the flowchart of FIG. 18, the feature amount data generation unit 12 will be described as the subject of an operation.

In FIG. 18, first, the feature amount data generation unit 12 acquires time-series data of sensor data related to the movement of both feet (step S101).

Next, the feature amount data generation unit 12 extracts gait waveform data for one gait cycle from the time-series data of the sensor data (step S102). The feature amount data generation unit 12 detects heel-contact and toe-off from the time-series data of the sensor data. The feature amount data generation unit 12 extracts time-series data of a section between consecutive heel-contacts as gait waveform data for one gait cycle.

Next, the feature amount data generation unit 12 normalizes the extracted gait waveform data for one gait cycle (step S103). The feature amount data generation unit 12 normalizes the gait waveform data for one gait cycle to a gait cycle of 0 to 100% (first normalization). The feature amount data generation unit 12 normalizes the ratio of the stance phase to the swing phase in the first normalized gait waveform data for one gait cycle to 60:40 (second normalization).

Next, the feature amount data generation unit 12 extracts a feature amount from the gait phase used for estimation of the pelvic inclination with respect to the normalized gait waveform (step S104). The feature amount data generation unit 12 extracts a feature amount to be used for estimation of the pelvic inclination.

Next, the feature amount data generation unit 12 generates a cluster feature amount (first feature amount) for each gait phase cluster using the extracted feature amount (step S105). In a case where the gait parameters are used for estimation of the pelvic inclination, the feature amount data generation unit 12 generates the gait parameters.

Next, the feature amount data generation unit 12 integrates the cluster feature amounts for each gait phase cluster to generate feature amount data for one gait cycle (step S106).

Next, the feature amount data generation unit 12 outputs the generated feature amount data to the pelvic inclination estimation device 13 (step S107).

[Pelvic Inclination Estimation Device]

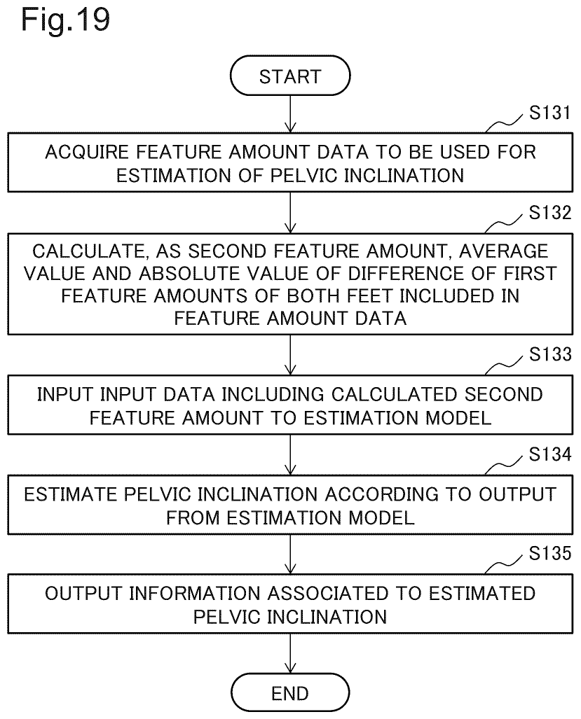

FIG. 19 is a flowchart for explaining the operation of the pelvic inclination estimation device 13. In the description according to the flowchart of FIG. 19, the pelvic inclination estimation device 13 will be described as the subject of an operation.

In FIG. 19, first, the pelvic inclination estimation device 13 acquires feature amount data to be used for estimation of the pelvic inclination from the measurement device 10 (step S131).

Next, the pelvic inclination estimation device 13 calculates, as the second feature amount, the average value of the first feature amounts included in the acquired feature amount data and the absolute value of the difference (step S132).

Next, the pelvic inclination estimation device 13 inputs input data including the calculated second feature amount to the estimation model for estimating the pelvic inclination (step S133).

Next, the pelvic inclination estimation device 13 estimates the pelvic inclination of the user according to the output (estimation value) from the estimation model (step S134). For example, the pelvic inclination estimation device 13 estimates a difference in the pelvic inclination of the user as the pelvic inclination of the user.

Next, the pelvic inclination estimation device 13 outputs information corresponding to the estimated pelvic inclination (step S135). For example, the pelvic inclination is output to a terminal device (not illustrated) carried by the user. For example, information corresponding to the pelvic inclination is output to a system that executes processing using the information.

(Application Example)

Next, an application example according to the present example embodiment will be described with reference to the drawings. In the following application example, an example in which the function of the pelvic inclination estimation device 13 installed in the mobile terminal carried by the user estimates the information related to the pelvic inclination using the feature amount data measured by the measurement device 10 arranged in the shoe will be described.

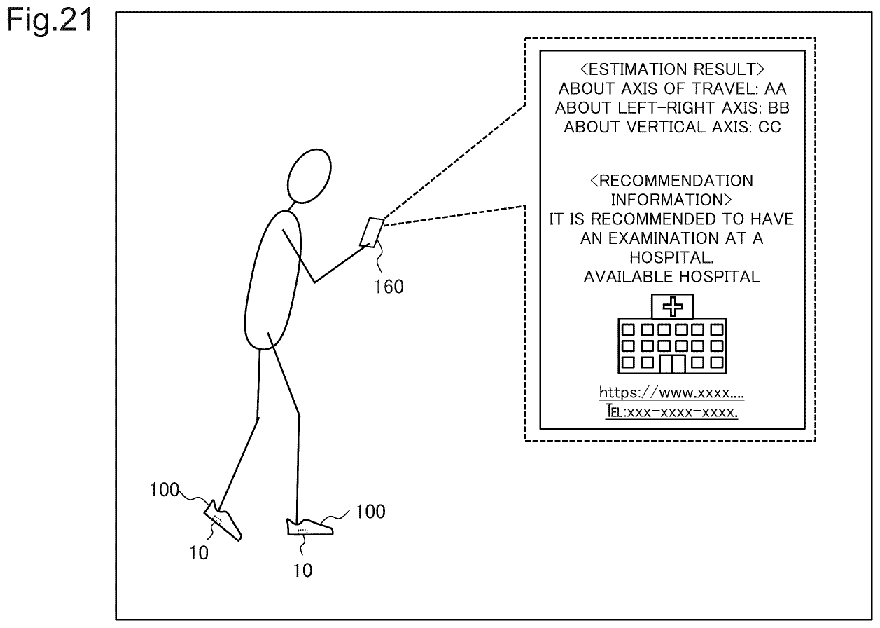

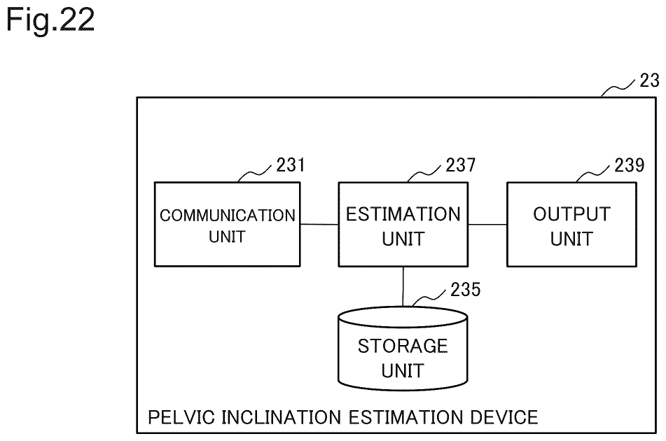

FIGS. 21 to 22 are conceptual diagrams illustrating an example in which the estimation result by the pelvic inclination estimation device 13 is displayed on the screen of the mobile terminal 160 carried by the user walking while wearing the shoe 100 on which the measurement device 10 is disposed. In the examples of FIGS. 21 to 22, information corresponding to the estimation result of the pelvic inclination using the feature amount data corresponding to the sensor data measured while the user is walking is displayed on the screen of the mobile terminal 160.

In the example of FIG. 21, the estimation results of the pelvic inclinations in the three axes of the traveling axis, the left-right axis, and the vertical axis are displayed on the display unit of the mobile terminal 160. In the example of FIG. 21, according to the estimation value of the pelvic inclination, recommendation information corresponding to the estimation result of the pelvic inclination such as “You should train the trunk.” is displayed on the display unit of the mobile terminal 160. In the example of FIG. 21, according to the estimation value of the pelvis inclination, recommendation information corresponding to the estimation result of “Training A is recommended. Please see the video below.” is displayed on the display unit of the mobile terminal 160. The user who has confirmed the information displayed on the display unit of the mobile terminal 160 can practice training for training the trunk by exercising with reference to the video of the training A according to the recommendation information.

In the example of FIG. 22, the estimation results of the pelvic inclinations in the three axes of the traveling axis, the left-right axis, and the vertical axis are displayed on the display unit of the mobile terminal 160. In the example of FIG. 22, recommendation information of “It is recommended to have an examination at a hospital.” is displayed on the display unit of the mobile terminal 160 according to the estimation value of the pelvic inclination. For example, a link destination or a telephone number to a hospital site where the user can be examined may be displayed on the screen of the mobile terminal 160. The user who has confirmed the information displayed on the display unit of the mobile terminal 160 can appropriately receive an examination of a disease related to the knee by visiting a hospital according to the recommendation information.

As described above, the estimation system of the present example embodiment includes the measurement device and the pelvic inclination estimation device. The measurement device is installed on the footwear of the subject to be estimated of the pelvic inclination which is an index related to the movement of the waist. The measurement device includes a sensor and a feature amount data generation unit. The sensor measures a spatial acceleration and a spatial angular velocity. The sensor generates sensor data related to the movement of the foot using the measured spatial acceleration and spatial angular velocity. The sensor outputs the generated sensor data. The feature amount data generation unit acquires time-series data of sensor data including features of a gait. The feature amount data generation unit extracts gait waveform data for one gait cycle from the time-series data of the sensor data. The feature amount data generation unit normalizes the extracted gait waveform data. The feature amount data generation unit extracts, from the normalized gait waveform data, a feature amount to be used for estimation of the pelvic inclination from a gait phase cluster including at least one temporally consecutive gait phase. The feature amount data generation unit generates feature amount data including the extracted feature amount. The feature amount data generation unit outputs the generated feature amount data to the pelvic inclination estimation device.

The pelvic inclination estimation device 23 includes a communication unit 231, a storage unit 235, an estimation unit 237, and an output unit 239.

The communication unit acquires feature amount data including a feature amount extracted from the gait waveforms of the spatial acceleration and the spatial angular velocity included in the sensor data related to the movement of the foot of the subject and used for estimation of the pelvic inclination which is an index related to the movement of the waist. The storage unit stores an estimation model that outputs an estimation value related to pelvic inclination according to an input of a feature amount included in the feature amount data. The estimation unit inputs the feature amount included in the acquired feature amount data to the estimation model, and estimates the pelvic inclination of the subject according to the estimation value related to the pelvic inclination output from the estimation model. The output unit outputs information corresponding to the pelvic inclination of the subject.

In the present example embodiment, the pelvic inclination, which is an index of the movement of the waist of the subject, is estimated using the feature amount extracted from the sensor data related to the movement of the foot of the subject. Therefore, according to the present example embodiment, it is possible to easily estimate the pelvic inclination, which is an index of the movement of the waist, with high accuracy in daily life.

In one aspect of the present example embodiment, the communication unit acquires feature amount data including gait parameters extracted from a gait waveform of a spatial acceleration and a spatial angular velocity included in sensor data. The storage unit stores an estimation model that outputs an estimation value related to pelvic inclination according to an input of gait parameters included in the feature amount data. The estimation unit inputs the gait parameters included in the acquired feature amount data to the estimation model, and estimates the pelvic inclination of the subject according to the estimation value related to the pelvic inclination output from the estimation model. According to the present aspect, the lumbar shake can be estimated with high accuracy using the feature amount data including the gait parameters.

In one aspect of the present example embodiment, the communication unit acquires the feature amount data including the first feature amount for each gait phase cluster extracted from the gait waveforms of the spatial acceleration and the spatial angular velocity included in the sensor data. The storage unit stores an estimation model that outputs an estimation value related to pelvic inclination according to an input of the first feature amount included in the feature amount data. The estimation unit inputs the first feature amount included in the acquired feature amount data to the estimation model, and estimates the pelvic inclination of the subject according to the estimation value related to the pelvic inclination output from the estimation model. According to the present aspect, the lumbar shake can be estimated with higher accuracy using the feature amount data including the first feature amount for each gait phase cluster.

A lumbar shake estimation device according to an aspect of the present example embodiment includes a calculation unit. The calculation unit calculates, as the second feature amount, an average value and a difference of the first feature amounts and the gait parameters used for estimation of the pelvic inclination among the first feature amounts and the gait parameters for both feet of the subject. The storage unit stores an estimation model that outputs an estimation value related to the pelvic inclination according to the input of the second feature amount. The estimation unit inputs the calculated second feature amount to the estimation model, and estimates the pelvic inclination of the subject according to the estimation value related to the pelvic inclination output from the estimation model. According to the present aspect, the lumbar shake can be estimated with higher accuracy using the average value/difference of the feature amounts for both feet.

In one aspect of the present example embodiment, the storage unit stores an estimation model that outputs an estimation value related to pelvic inclination according to the attribute of the subject and the input of the second feature amount. The estimation unit inputs the attribute of the subject and the second feature amount to the estimation model, and estimates the pelvic inclination of the subject according to the estimation value related to the pelvic inclination output from the estimation model. According to the present aspect, the lumbar shake can be estimated with higher accuracy using the attribute of the subject.

In one aspect of the present example embodiment, the storage unit stores an estimation model that outputs an estimation value related to pelvic inclination according to an input of a feature amount included in the feature amount data. The estimation model outputs a variation width of at least one pelvic inclination related to three axes of a traveling axis, a left-right axis, and a vertical axis in one gait cycle as an estimation value related to the pelvic inclination. The estimation unit inputs the feature amount included in the acquired feature amount data to the estimation model, and estimates the pelvic inclination of the subject according to the variation width of at least one of the pelvic inclinations in the three axes of the traveling axis, the left-right axis, and the vertical axis output from the estimation model. According to the present aspect, the lumbar shake can be estimated with higher accuracy according to the variation width of the lumbar shake with respect to the three axes of the traveling axis, the left-right axis, and the vertical axis.

In one aspect of the present example embodiment, the pelvic inclination estimation device is mounted on a terminal device having a screen visually recognizable by the subject. The pelvic inclination estimation device displays information related to the pelvic inclination estimated according to the movement of the foot of the subject on the screen of the terminal device. According to the present aspect, the information related to the pelvic inclination estimated for the subject can be accurately presented to the subject.

The pelvic inclination is an index of physical conditions and health conditions about the waist. For example, the degree of postoperative recovery of the disc hemiation can be determined according to the angle of the pelvic inclination. For example, the angle of the sagittal/coronal plane can be used for evaluating the degree of waist bending. The angle in the sagittal plane corresponds to an inclination angle about the left-right axis. The angle in the coronal plane corresponds to the angle of inclination about the axis of travel. It is normal if the angle of the sagittal/coronal plane during walking is within 5 degrees. When the angle of the sagittal plane/coronal plane during walking is 5 to 15 degrees, it is a level requiring attention. If the angle of the sagittal/coronal plane during walking is equal to or more than 15 degrees, it is a level where the user needs someone's help. For example, the angle of the sagittal/coronal plane can be used for evaluating the posture during walking. If the score of the posture according to the angle of the sagittal plane/coronal plane is set, the posture can be evaluated according to the score. For example, the inclination of the pelvis inclination about the left-right axis corresponding to the angle in the coronal plane becomes a check item of osteoarthritis of the pelvis and lateral inclination (rising/lowering). The pelvic inclination about the left-right axis is related to left-right balance during walking. Therefore, the pelvic inclination about the left-right axis is an index of walking stability.

For example, if the person cannot walk in a flexible walking posture due to knee arthropathy, the sinking pattern of the body immediately after the foot touches the ground becomes clumsy, and the pelvic inclination in the vertical direction is affected. Therefore, the pelvic inclination in the vertical direction becomes an index of the state and progress of knee arthropathy. For example, if a person has hemiplegia, the body sinks down and the pelvic inclination in the vertical direction is affected at the timing of landing with the foot on the side of the half body where the power is not applied. Therefore, the pelvic inclination in the vertical direction becomes an index of the state and the degree of progress of hemiplegia. For example, symptoms such as lumbar spinal stenosis, neurogenic intermittent claudication, and degenerative lumbar spondylolisthesis are also associated with pelvic inclination.

### Second Example Embodiment

Next, a pelvic inclination estimation device according to a second example embodiment will be described with reference to the drawings. The pelvic inclination estimation device of the present example embodiment has a simplified configuration of the pelvic inclination estimation device of the first example embodiment.

FIG. 22 is a block diagram illustrating an example of a configuration of the pelvic inclination estimation device 23 according to the present example embodiment. The pelvic inclination estimation device 23 includes a communication unit 231, a storage unit 235, an estimation unit 237, and an output unit 239.

The communication unit 231 acquires feature amount data including a feature amount extracted from the gait waveforms of the spatial acceleration and the spatial angular velocity included in the sensor data related to the movement of the foot of the subject and used for estimation of the pelvic inclination which is an index related to the movement of the waist. The storage unit 235 stores an estimation model that outputs an estimation value related to the pelvic inclination according to the input of the feature amount included in the feature amount data. The estimation unit 237 inputs the feature amount included in the acquired feature amount data to the estimation model, and estimates the pelvic inclination of the subject according to the estimation value related to the pelvic inclination output from the estimation model. The output unit 239 outputs information corresponding to the pelvic inclination of the subject.

In the present example embodiment, the pelvic inclination, which is an index of the movement of the waist of the subject, is estimated using the feature amount extracted from the sensor data related to the movement of the foot of the subject. Therefore, according to the present example embodiment, it is possible to easily estimate the pelvic inclination, which is an index of the movement of the waist, with high accuracy in daily life.

(Hardware)

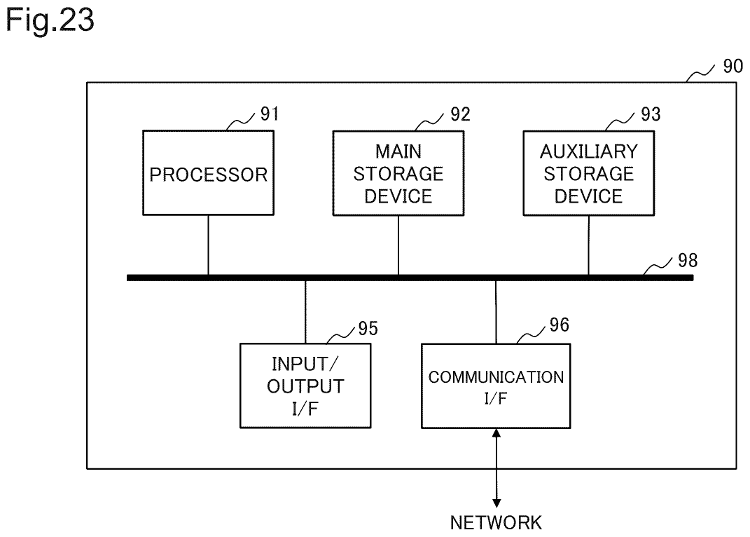

Here, a hardware configuration for executing the processing according to each example embodiment of the present disclosure will be described using the information processing device (computer) 90 of FIG. 23 as an example. The information processing device 90 in FIG. 23 is a configuration example for executing the processing of each example embodiment, and does not limit the scope of the present disclosure.

As illustrated in FIG. 23, the information processing device 90 includes a processor 91, a main storage device 92, an auxiliary storage device 93, an input/output interface 95, and a communication interface 96. In FIG. 23, the interface is abbreviated as an interface (I/F). The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program (instruction) stored in the auxiliary storage device 93 or the like in the main storage device 92. For example, the program is a software program for executing the processing of each example embodiment. The processor 91 executes the program developed in the main storage device 92. The processor 91 executes the processing according to each example embodiment by executing the program.

The main storage device 92 has an area in which a program is developed. A program stored in the auxiliary storage device 93 or the like is developed in the main storage device 92 by the processor 91. The main storage device 92 is, for example, a volatile memory such as a dynamic random access memory (DRAM). A nonvolatile memory such as a magnetoresistive random access memory (MRAM) may be configured and added as the main storage device 92.

The auxiliary storage device 93 stores various types of data such as programs. The auxiliary storage device 93 is a local disk such as a hard disk or a flash memory. Various types of data may be stored in the main storage device 92, and the auxiliary storage device 93 may be omitted.

The input/output interface 95 is an interface for connecting the information processing device 90 and a peripheral device based on a standard or a specification. The communication interface 96 is an interface for connecting to an external system or device through a network such as the Internet or an intranet based on a standard or a specification. The input/output interface 95 and the communication interface 96 may be shared as an interface connected to an external device.

Input devices such as a keyboard, a mouse, and a touch panel may be connected to the information processing device 90 as necessary. These input devices are used for inputting information and settings. When a touch panel is used as the input device, a screen having a touch panel function serves as an interface. The processor 91 and the input device are connected via the input/output interface 95.

The information processing device 90 may be provided with a display device for displaying information. In a case where a display device is provided, the information processing device 90 includes a display control device (not illustrated) for controlling display of the display device. The information processing device 90 and the display device are connected via the input/output interface 95.

The information processing device 90 may be provided with a drive device. The drive device mediates reading of data and a program stored in a recording medium and writing of a processing result from the information processing device 90 to the recording medium between the processor 91 and the recording medium (program recording medium). The information processing device 90 and the drive device are connected via an input/output interface 95.

The above is an example of a hardware configuration for enabling the processing according to each example embodiment of the present invention. The hardware configuration of FIG. 23 is an example of a hardware configuration for executing the processing according to each example embodiment, and does not limit the scope of the present invention. A program for causing a computer to execute processing according to each example embodiment is also included in the scope of the present invention.

A program recording medium in which the program according to each example embodiment is recorded is also included in the scope of the present invention. The recording medium can be achieved by, for example, an optical recording medium such as a compact disc (CD) or a digital versatile disc (DVD). The recording medium may be a semiconductor recording medium such as a universal serial bus (USB) memory or a secure digital (SD) card. The recording medium may be a magnetic recording medium such as a flexible disk, or another recording medium. In a case where the program executed by the processor is recorded in the recording medium, the recording medium corresponds to a program recording medium.

The components of the example embodiments may be arbitrarily combined. The components of the example embodiments may be implemented by software. The components of each example embodiment may be implemented by a circuit.

The previous description of embodiments is provided to enable a person skilled in the art to make and use the present invention. Moreover, various modifications to these example embodiments will be readily apparent to those skilled in the art, and the generic principles and specific examples defined herein may be applied to other embodiments without the use of inventive faculty. Therefore, the present invention is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. A pelvic inclination estimation device comprising:
a memory storing instructions, and
a processor connected to the memory and configured to execute the instructions to:
acquire feature amount data including a feature amount to be used for estimation of a pelvic inclination that is an index related to movement of a waist, the feature amount being extracted from a gait waveform of a spatial acceleration and a spatial angular velocity included in sensor data related to movement of a foot of a user;
input the feature amount included in the acquired feature amount data to an machine learning model that outputs an estimation value related to the pelvic inclination in response to an input of the feature amount included in the feature amount data;
estimate a pelvic inclination of the user according to the estimation value related to the pelvic inclination output from the machine learning model; and
display information regarding a hospital at which the user can seek medical advice according to the estimation result of the pelvic inclination of the user on a screen of a mobile terminal used by the user.

2. The pelvic inclination estimation device according to claim 1, wherein
the machine learning model is trained to output the estimation value related to the pelvic inclination according to an input of the gait parameter included in the feature amount data,
the processor is configured to execute the instructions to
acquire the feature amount data including gait parameters extracted from a gait waveform of the spatial acceleration and the spatial angular velocity included in the sensor data,
input the gait parameters included in the acquired feature amount data to the machine learning model, and
estimate the pelvic inclination of the user according to the estimation value related to the pelvic inclination output from the machine learning model.

3. The pelvic inclination estimation device according to claim 2, wherein
the machine learning model is trained to output an estimation value related to the pelvic inclination according to an input of the first feature amount included in the feature amount data,
the processor is configured to execute the instructions to
acquire the feature amount data including a first feature amount for each gait phase cluster extracted from the gait waveform of the spatial acceleration and the spatial angular velocity included in the sensor data,
input the first feature amount included in the acquired feature amount data to the machine learning model, and
estimate the pelvic inclination of the user according to the estimation value related to the pelvic inclination output from the machine learning model.

4. The pelvic inclination estimation device according to claim 3, wherein
the machine learning model is trained to output an estimation value related to the pelvic inclination according to an input of the first feature amount included in the feature amount data,
the processor is configured to execute the instructions to
calculate, as a second feature amount, an average value and a difference of the first feature amounts and the gait parameters used for estimation of the pelvic inclination among the first feature amounts and the gait parameters for both feet of the user,
input the calculated second feature amount to the machine learning model, and
estimate the pelvic inclination of the user according to the estimation value related to the pelvic inclination output from the machine learning model.

5. The pelvic inclination estimation device according to claim 4, wherein
the machine learning model is trained to output an estimation value related to the pelvic inclination according to an input of an attribute of the user and the second feature amount,
the processor is configured to execute the instructions to
input the attribute of the user and the second feature amount input to the machine learning model, and
estimate the pelvic inclination of the user according to the estimation value related to the pelvic inclination output from the machine learning model.

6. The pelvic inclination estimation device according to claim 1, wherein
the machine learning model is trained to output at least one variation width of the pelvic inclination related to three axes of a traveling axis, a left-right axis, and a vertical axis in one gait cycle as an estimation value related to the pelvic inclination according to the input of the feature amount included in the feature amount data,
the processor is configured to execute the instructions to
input a feature amount included in the acquired feature amount data to the machine learning model, and
estimate the pelvic inclination of the user according to a variation width of at least one of the pelvic inclinations in the three axes of the traveling axis, the left-right axis, and the vertical axis output from the machine learning model.

7. The pelvic inclination estimation device according to claim 1, wherein
the processor is configured to execute the instructions to
display recommendation information according to the estimation result of the pelvic inclination of the user on the screen of the mobile terminal used by the user with content optimized for healthcare application.

8. An estimation system comprising:
the pelvic inclination estimation device according to claim 1; and
a measurement device including a sensor that measures a spatial acceleration and a spatial angular velocity, and generates the sensor data based on the spatial acceleration and the spatial angular velocity, and configured to generate feature amount data including a feature amount used for estimating a pelvic inclination using the sensor data.

9. An estimation method executed by a computer, the method comprising:
acquiring feature amount data including a feature amount to be used for estimation of a pelvic inclination that is an index related to movement of a waist, the feature amount being extracted from a gait waveform of a spatial acceleration and a spatial angular velocity included in sensor data related to movement of a foot of a user;
inputting the feature amount included in the acquired feature amount data to an machine learning model that outputs an estimation value related to the pelvic inclination in response to an input of the feature amount included in the feature amount data;
estimating a pelvic inclination of the user according to the estimation value related to the pelvic inclination output from the machine learning model; and
displaying information regarding a hospital at which the user can seek medical advice according to the estimation result of the pelvic inclination of the user on a screen of a mobile terminal used by the user.

10. A non-transitory program recording medium recorded with a program causing a computer to perform the following processes:
acquiring feature amount data including a feature amount to be used for estimation of a pelvic inclination that is an index related to movement of a waist, the feature amount being extracted from a gait waveform of a spatial acceleration and a spatial angular velocity included in sensor data related to movement of a foot of a user;
inputting the feature amount included in the acquired feature amount data to an machine learning model that outputs an estimation value related to the pelvic inclination in response to an input of the feature amount included in the feature amount data;
estimating a pelvic inclination of the user according to the estimation value related to the pelvic inclination output from the machine learning model; and
displaying information regarding a hospital at which the user can seek medical advice according to the estimation result of the pelvic inclination of the user on a screen of a mobile terminal used by the user.
